SEQUENCE LISTING (4 sequences)

SEQ ID NO: 1 - protein, 9 aa, Homo sapiens
PVNFKFLSH

SEQ ID NO: 2 - protein, 11 aa, Homo sapiens
VDPVNFKFLSH

SEQ ID NO: 3 - protein, 12 aa, Homo sapiens
RVDPVNFKFLSH

SEQ ID NO: 4 - protein, 9 aa, Homo sapiens
PVNFKWLSH

(19) United States
(12) Patent Application Publication
(10) Pub. No.: US 2014/0296153 A1
(43) Pub. Date: Oct. 2, 2014

(54) Oligodendrocyte Differentiation

(71) Applicants: Sara Alves Xapeli, Coimbra (PT); João José Oliveira Malva, Coimbra (PT); Ricardo Augsto de Melo Reis, Rio de Janeiro (BR)

(72) Inventors: Sara Alves Xapeli, Coimbra (PT); João José Oliveira Malva, Coimbra (PT); Ricardo Augsto de Melo Reis, Rio de Janeiro (BR)

(21) Appl. No.: 14/232,100

(22) PCT Filed: Aug. 8, 2012
(86) PCT No.: PCT/GB2012/051917

§ 371 Date: Jun. 10, 2014

(30) Foreign Application Priority Data
Aug. 8, 2011 (GB) 1113596.9

(51) Int. Cl. C07K 7/06

(52) U.S. Cl. CPC C07K 7/06
USPC 514/17.8; 530/328; 514/17.7; 435/377; 514/17.9

(57) ABSTRACT

The present invention provides compounds, compositions and methods for treatment and/or prevention of neurodegenerative diseases, including but not limited to autoimmune diseases, such as multiple sclerosis, in which demyelination, (the loss of the myelin sheath that insulates the nerves) is an associated or causative feature. The data provided demonstrate the utility of the compounds and compositions according to this invention to promote oligodendrogenesis and myelination or remyelination.

### FIELD OF THE INVENTION

Compounds, compositions and methods are disclosed for use in inducing/promoting myelin production and differentiation of pre-oligodendrocytes (oligodendrocyte precursor cells, OPCs) into oligodendrocytes. Also disclosed is the use of such compounds, compositions, oligodendrocytes and combinations thereof to treat neurodegenerative diseases such as multiple sclerosis (MS).

### BACKGROUND OF THE INVENTION

A demyelinating disease is characterized by the loss of myelin sheaths around axons, which are important to ensure the high-speed conduction of nervous impulses. In the central nervous system (CNS), demyelination is usually the consequence of a direct or indirect insult to the population of cells known oligodendrocyte, which make and maintain the myelin sheath. When such insult occurs, due to the lack of axonal insulation by the myelin sheath, neuronal communication becomes deficient, therefore leading to brain function impairments (in sensation, movement and/or cognition). Some demyelinating diseases are caused either by a viral infection, a genetic abnormality or inflammatory damage. One of the most significant of these is multiple sclerosis (MS).

The myelin sheaths can be re-generated in demyelinated axons by a process called remyelination. In early phases of a demyelinating disorder, endogenous oligodendrocyte precursor cells (OPCs) spontaneously remyelinate newly nude axons in damaged areas. However, as disease progresses, the efficiency of remyelination decreases (reviewed by Franklin and French-Constant, 2008 in ref. [9]).

Although these neurological disorders are a common cause of disability in young adults, so far, there is no effective treatment against them. However, it has been suggested that neural stem cells (NSCs) might be an endogenous source of new oligodendrocytes for use in regenerative medicine concerning myelin pathologies. NSCs are multipotent cells that can self-renew and differentiate into all neural cell types, i.e. neurons, astrocytes and oligodendrocytes. These multipotent cells are present in the adult mammalian brain and are restricted to specialized niches, with the subventricular zone (SVZ) lining the lateral ventricles being the most extensive niche identified to date. (Reviewed by Gonzalez-Perez and Alvarez-Buylla, 2011 in ref. [13]).

In the SVZ, new neurons that arise travel via the rostral migratory stream (RMS) to the olfactory bulb (OB), where they differentiate into functional interneurons [16], contributing to odour memory and discrimination [5]. In vivo, the adult SVZ appears to be mainly neurogenic in normal conditions [17]. In fact, compared with the large number of new neurons which arise in the SVZ, few oligodendrocytes are normally produced from adult SVZ [18]. However, upon brain injury or disease, several studies indicate that cells from the SVZ have the capacity to undergo neurogenesis and gliogenesis, depending on the damage [21]. In models of experimental demyelination and multiple sclerosis, reactivation of the SVZ increases proliferation and induces oligodendrogenesis to some extent [19].

Moreover, data exists which suggests that NSCs transplantated into the CNS can produce oligodendrocyte progenitors that can differentiate into mature and functional myelinating oligodendrocytes [3, 7, 22, 24].

Therefore, since the SVZ contains multipotential NSCs which are able to self-renew, migrate extensively and differentiate, this structure is of great interest to promote repair of the diseased brain. However, mobilization, differentiation and replacement remain limited, implying that compounds, compositions and methods which can promote these phenomena represent a long-felt and as yet unmet need.

Interestingly, endocannabinoids have emerged as a potential target to modulate oligodendrogenesis. Cannabinoids act on at least 2 types of receptors, CB₁R and CB₂R, which are predominantly distributed in the CNS and immune system, respectively. In the brain, CB₁R are targeted by endocannabinoids such as anandamide and 2-arachidonylglycerol, which are molecules generated “on demand” by cleavage of plasma membrane lipid precursors [10]. Several reports have provided data to suggest that endocannabinoids may have a role in oligodendrogenesis. In fact, Arevalo-Martin and collaborators [4] have found that post-natal treatment with a CB₁R agonist increased the number of oligodendrocyte transcription factor 2 (Olig2)-positive cells in the SVZ of rats. Moreover, endocannabinoids have also been suggested to play a role in oligodendrocyte differentiation in more recent studies [12, 25].

As described in [15], Hemopressin (Hp), a 9 residue-long peptide derived from Hemoglobin (Hb), has been identified as a peptide ligand that selectively binds CB₁R cannabinoid receptors. It has been described as a CB₁R receptor selective antagonist because it is able to efficienctly block signalling by CB₁R receptors but not by other members of G protein coupled receptors, including CB₂R. Hp also behaves as an inverse antagonist of CB₁R as it is able to block constitutive activity of these receptors to the same extend as the antagonist rimonabant [15].

The finding that Hp functions as an inverse agonist as well as an antagonist of CB₁R [8, 15] suggests that cannabinoid receptor activity could be modulated by peptides derived from Hb. A recent study has identified N-terminally extended forms of Hp containing either 3 or 2 additional amino acids (RVD-Hp-alpha or VD-Hp-alpha) in mouse brain extracts which, in contrast to Hp, function as agonists [11]. It has also been reported that neurons and oligodendrocytes express Hb [6, 23]. WO 2011/011847 (incorporated herein by reference) discloses the use of Hp for the treatment of obesity and/or diabetes.

Gomes et al. [28] provide a review of Hemoglobin-derived Peptides as Novel Type of Bioactive Signaling Molecules, and cites several references which are likewise of interest (including, e.g. Biagioli et al reference from PNAS 2009).

Gomez et al. [12] describe that the CB₁R and CB₂R antagonists rimonabant and AM630 do not induce but impair OPC differentiation into mature oligodendrocytes. We believe that the present patent disclosure represents the first instance in which it is demonstrated that Hp and related compounds, as defined herein, can play a direct role in the induction of differentiation of NSCs, OPCs, and, specifically, cells derived from the SVZ, into oligodendrocytes.

Oligodendrocytes (OGs) assemble the myelin sheath around axons in the central nervous system. The control of oligodendrogenesis (the formation of oligodendrocytes) and differentiation of neural precursor cells (NPCs) into mature oligodendrocytes is thus desirable in many neurological disorders. One example is multiple sclerosis (MS), a demyelinating disease which has been described as an inflammatory neurodegenerative disorder. MS affects more than one million people worldwide and is the leading cause of neurological (Usability in young adults. MS is associated with the destruction of myelin, oligodendrocytes and axons localized to chronic lesions. The demyelination observed in MS is not always permanent and remyelination has been documented in early stages of the disease. Remyelination of neurons requires oligodendrocytes.

The present invention is thus aimed at providing an effective treatment of neurodegenerative diseases.

### SUMMARY OF THE INVENTION

The present disclosure provides a novel solution to the long-felt and unmet need in the art by surprisingly showing that it is possible to use isolated Hb-derived peptides, such as Hp and related compounds, and compositions comprising these molecules to stimulate NSCs, NPCs, OPCs, or other oligodendrocyte progenitor cells, such as those derived from the SVZ, to undergo oligodendrogenesis.

This invention disclosure provides compositions, methods and means for utilizing Hb-derived peptides, in particular Hp and variants thereof, in new cellular and pharmacological-based strategies to restore/regenerate neural function in a mammal and in particular to treat and/or prevent demyelinating diseases.

The present invention provides a peptide according to SEQ ID No 1 or a variant thereof for use as a neuromodulating agent. In particular, the invention relates to the use of a peptide according to SEQ ID No 1 or a variant thereof for use as a neuromodulating agent in the treatment of a neurodegenerative disorder.

The peptide may be formulated as part of a pharmaceutical composition. Thus, the present invention also provides a pharmaceutical composition which comprises as an active ingredient, either (i) a compound which is variant of Hb, particularly Hp, (ii) a cell treated with the compound defined in (i), or (iii) a combination of (i) and (ii), not necessarily administered at the same time or in the same composition of matter or dosage, but as part of an unitary treatment regimen. Pharmaceutically acceptable vehicles known in the art are included for induction of myelination or remyelination and treatment or prevention of diseases in patients in need of such treatment. Such vehicles include, but are not limited to, saline solutions, iso-osmotic compositions and the like which are non-toxic and which preserve peptides, proteins, and/or cells in viable condition to exert their desired physiologic effect. Where cells are utilized in vivo, these are preferably non-immunogenic compositions. In a preferred embodiment according to this invention, NSCs, NPCs, OPCs, and/or cells derived from the SVZ are harvested from a subject, treated in vitro/ex vivo with an active compound according to this invention in order to induce such cells to differentiate into oligodendrocytes. The thus treated cells from the subject are then re-introduced into the subject at a physiologic site such that the oligodendrocytes initiate myelination or re-myelination of neuronal tissue in need thereof. The present invention thus refers to compounds and compositions, which comprise a peptide according to SEQ ID No. 1 a variant thereof for contact with NSCs, NPCs, OPCs, and the like, including but not limited to cells derived from the SVZ, in vivo, or in vitro. This contact induces such cells to differentiate into oligodendrocytes. When such cells are introduced into said patients, they contribute toward myelination or remyelination and treatment or prevention of diseases associated with demyelination.

In one embodiment according to this invention, a G protein-coupled receptor that is targeted is a cannabinoid receptor and in particular the CB₁ receptor. Preferably, said active ingredient is the peptide hemopressin, with the following amino acid sequence PVNFKFLSH (proline-valine-asparagine-phenylalanine-lysine-phenylalanine-leucine-serine-histidine) SEQ. ID. 1, or variant thereof. The pharmaceutically acceptable vehicle is preferably a sterile iso-osmotic solution with the same osmotic pressure of an isotonic solution of blood and that is compatible with the active ingredient.

The invention provides a pharmaceutical composition which may be used in the treatment of neurodegenerative diseases, such as those associated with demyelination. Furthermore, this invention demonstrates that said pharmaceutical composition can effectively achieve said results through an administrative route including but not limited to orally, intraperitoneally, intravenously, and intrathecally, where the active compounds are introduced in vivo, or are used ex vivo or in vitro to treat cells which are thereby induced to differentiate into oligodendrocytes.

Accordingly, it is an object of this invention to provide a process for the treatment of neuronal precursor/stem cells with Hp and/or other Hb-derived peptides to induce oligodendrogenesis.

A further object of this invention is to provide a new method, compounds and compositions for treatment and/or prevention of neurodegenerative, including demyelinating or dysmyelinating diseases.

A further object of this invention is to provide a process for inducing oligodendrogenesis from neural progenitor/stem cells based on Hp and/or other Hb-derived peptides.

A further object of the invention is to provide a pharmaceutical composition comprising an active ingredient, a variant thereof, which acts as an antagonist or inverse agonist of cannabinoid type 1 receptors and a pharmaceutically acceptable vehicle, characterized by the fact that administration of said pharmaceutical composition improves, prevents and treats conditions associated with demyelination by inducing pre-oligodendrocytes to differentiate into oligodendrocytes, which are active in myelination.

Further objects and benefits of this invention will be appreciated by those skilled in the art from a review of the complete disclosure and the claims which follow.

### BRIEF DESCRIPTION OF THE DRAWINGS

FIG. 1—SVZ cells express CB1R. Representative fluorescent confocal photomicrographs depicting CB₁R (green staining) and nestin (A) or GFAP (B) (red staining for both) immunoreactivity in SVZ cells migrating out of a neurosphere 7 days after plating. Hoechst 33342 staining (blue) was used to visualize cell nuclei. Scale bars: 20 μm.

FIG. 2—Hp does not induce cell proliferation or cell death in subventricular zone (SVZ) cell cultures. (A): Representative confocal photos of cell nuclei in SVZ cell cultures maintained for 48 hours in the absence (control) or in the presence of 100 nM or 1 μM Hp and immuno-labeled for BrdU (red nuclei). (B): Percentage of BrdU-immunostained nuclei. (C): Representative fluorescence photos of cell nuclei in a control and in a Hp-treated SVZ culture stained using the Tunel method (green nuclei). (D): Percentages of Tunel-stained nuclei in cultures maintained for 48 hours in the absence (control) or in the presence of Hp. Abbreviations: BrdU, 5-bromo-2′-deoxyuridine; Tunel, terminal deoxynucleotidyl transferase dUTP nick-end labeling.

FIG. 3—(A): Number of primary neurospheres in SVZ cell cultures grown in SFM containing 5 ng/ml EGF and 2.5 ng/ml FGF-2 and supplemented or not (control) with 1 μM Hp or 50 ng.ml⁻¹ HGF. (B): Primary neurospheres obtained in control, Hp and HGF conditions were collected, dissociated as single cells and replated in SFM containing 5 ng/ml EGF and 2.5 ng/ml FGF-2 to allow formation of secondary neurospheres. The numbers of secondary neurospheres obtained in each condition are depicted. Mean±SEM of six independent experiments are represented. *2<0.05 and *2<0.01 using unpaired Student's t-test for comparison with SVZ neurospheres control cultures

FIG. 4—Hp induces oligodendrocyte differentiation in mouse SVZ cell cultures. (A): Bar graph depicts the percentages of oligodendrocyte-like responding cells in SVZ control cultures and in cultures exposed to Hp and AM251 (CB1R antagonist) for 7 days. ***P<0.001 using unpaired Student's t-test for comparison with SVZ control cultures. (B): Representative single cell calcium imaging profiles of response of about 100 cells in a control culture, in a culture treated with 1 μM Hp and in a culture co-treated with Hp 1 μM and AM251 1 μM for 7 days. SVZ cultures were perfused continuously with Krebs solution and stimulated with 50 mM KCl, with 100 μM Histamine and with 0.1 U/ml thrombin.

FIG. 5—Hp induces oligodendrocyte differentiation in mouse SVZ cell cultures. (A): Left pannel: Bar graph depicts the percentages relative to control of Olig2 protein levels normalized to β-actin, in SVZ control cultures and in cultures exposed to Hp for 7 days. *2<0.05 using Dunnett's Multiple Comparison Test for comparison with SVZ control cultures. Right panel: Representative western blot of Olig2 (37 KDa) and β-actin (42 KDa) protein levels from SVZ control cultures and from cultures exposed to Hp. (B): Immunocytochemistry for Olig2 under control and Hp treated conditions.

### DETAILED DISCLOSURE OF THE PREFERRED EMBODIMENTS ACCORDING TO THIS INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of neuroscience, tissue culture, molecular biology, chemistry and biochemistry, which are within the skill of the art. Such techniques are explained fully in the literature.

The hallmark of some neurodegenerative autoimmune, inflammatory or traumatic diseases, including but not limited to multiple sclerosis, transverse myelitis, Devic's disease, progressive multifocal leukoencephalopathy, Optic neuritis, Leukodystrophies, Guillain-Barré syndrome, Charcot-Marie-Tooth disease, spinal cord injury, is demyelination, which is the loss of the myelin sheath that insulates the nerves. Presently, treatments for diseases that affect the myelin sheaths fail to prevent long-term motor and cognitive decline in patients. Therefore, it is of great importance to find substances that will be able to promote oligodendrogenesis and remyelination.

The present invention demonstrates the utility of hemopressin, an alpha hemoglobin fragment originally identified in extracts of rat brain using an enzyme capture technique and defined by the amino acid sequence PVNFKFLSH (proline-valine-asparagine-phenylalanine-lysine-phenylalanin e-leucine-serine-histidine), SEQ. ID. 1, or a biologically active variant thereof, in the induction of oligodendrogenesis.

We show herein that Hp (a peptide recently shown to act on the endocannabinoid CB₁ receptor) promotes oligodendrogenesis. We also show that this peptide does not induce proliferation or increase cell death, but it increases oligodendrocyte differentiation in cultured subventricular zone (SVZ) neural stem/progenitor cells derived from the neonatal P1-3 C57BL/6 mice. Oligodendrocyte Progenitor Cells (OPCs) from other sites, and in general, NPCs, above and beyond those found in or derived from the SVZ, may likewise be induced to differentiate into oligodendrocytes, using the methods, use, compounds and compositions according to this invention, including in vivo and in vitro. The mammalian sub-ventricular zone (SVZ) is the largest germinative zone of the adult brain, which contains a well characterized stem cell niche. Neural stem cells from the SVZ give rise to progenitor cells which have the capacity to differentiate into a number of cells types of the CNS, including the myelin-forming oligodendrocytes. Experimental models of demyelination in rodent have demonstrated enhanced proliferation and recruitment of SVZ progenitors into myelin lesions, in response to demyelination. Moreover, cell lineage tracing experiments have shown that SVZ progenitor cells can give rise to oligodendrocytes in demyelinated lesions, that could potentially contribute to remyelination. Methods for functional identification of SVZ-derived oligodendrocytes that can be used are described in WO 2010/046876 incorporated herein by reference.

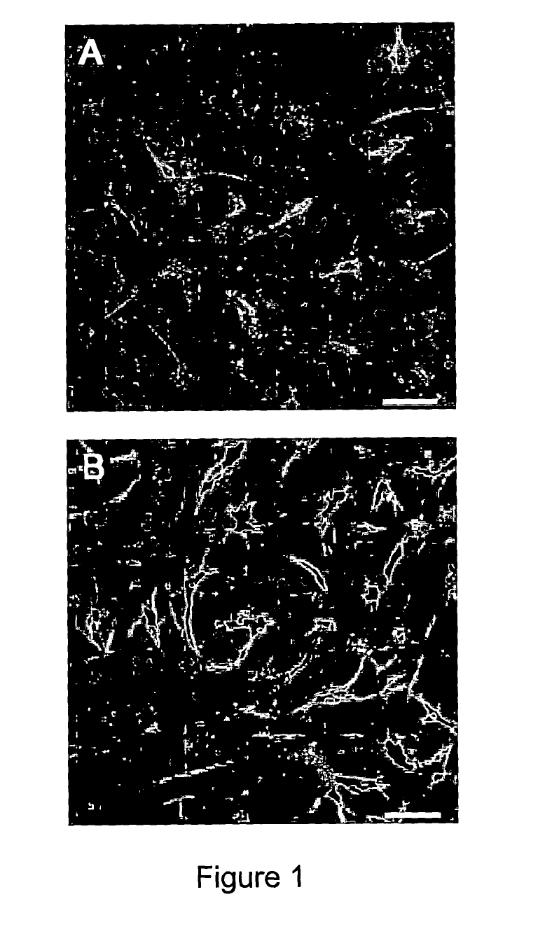

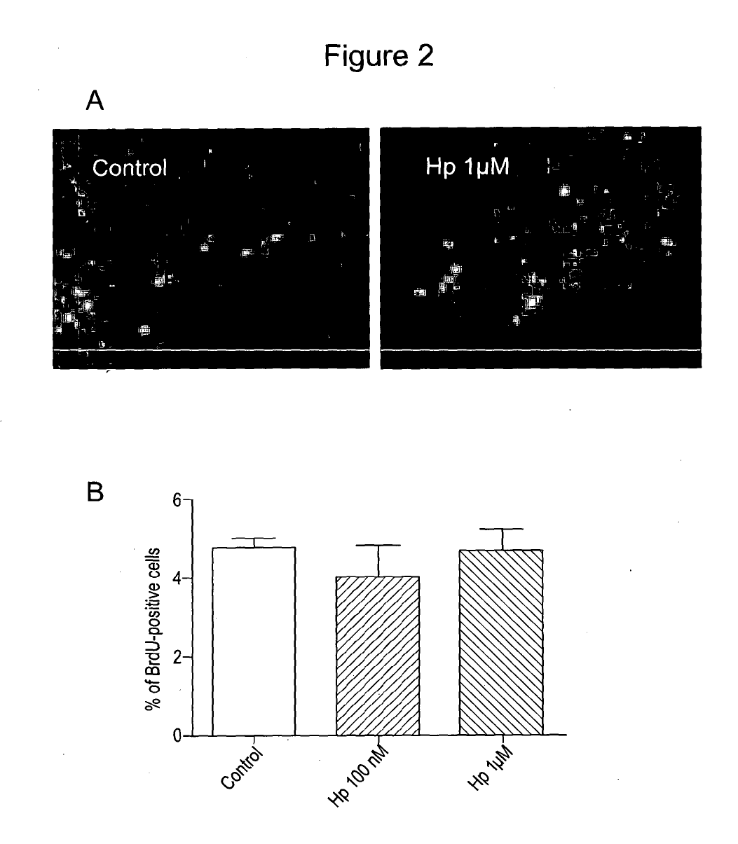

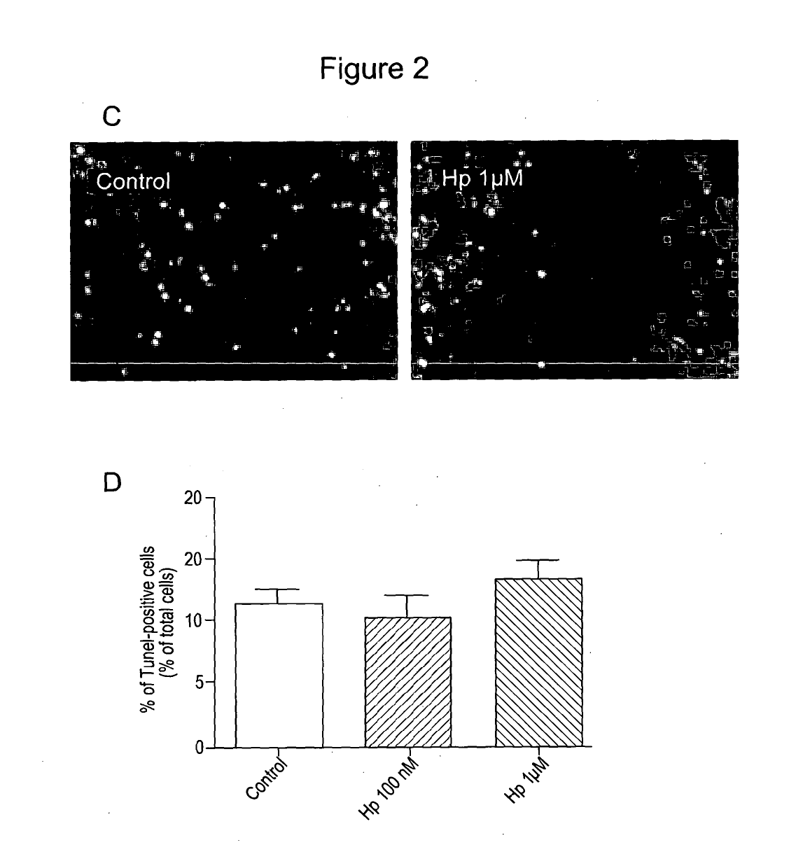

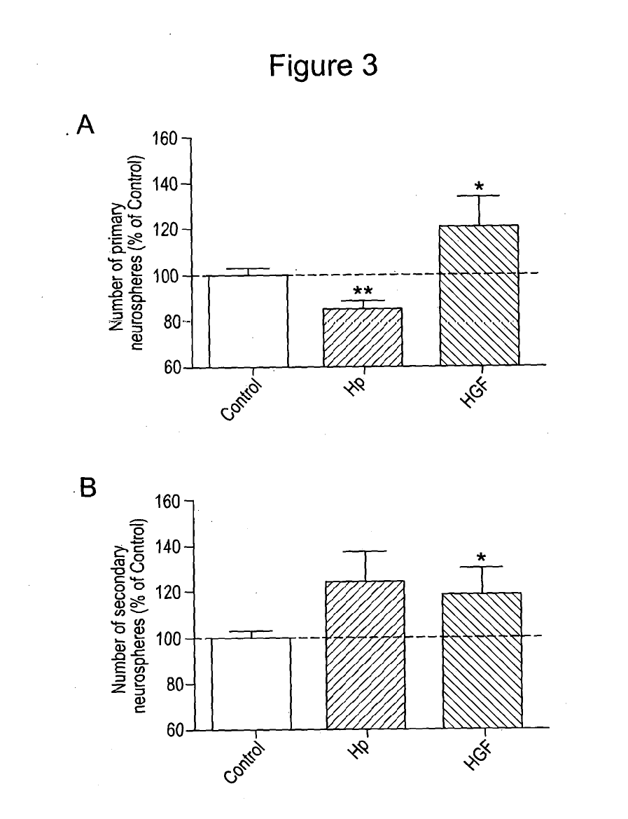

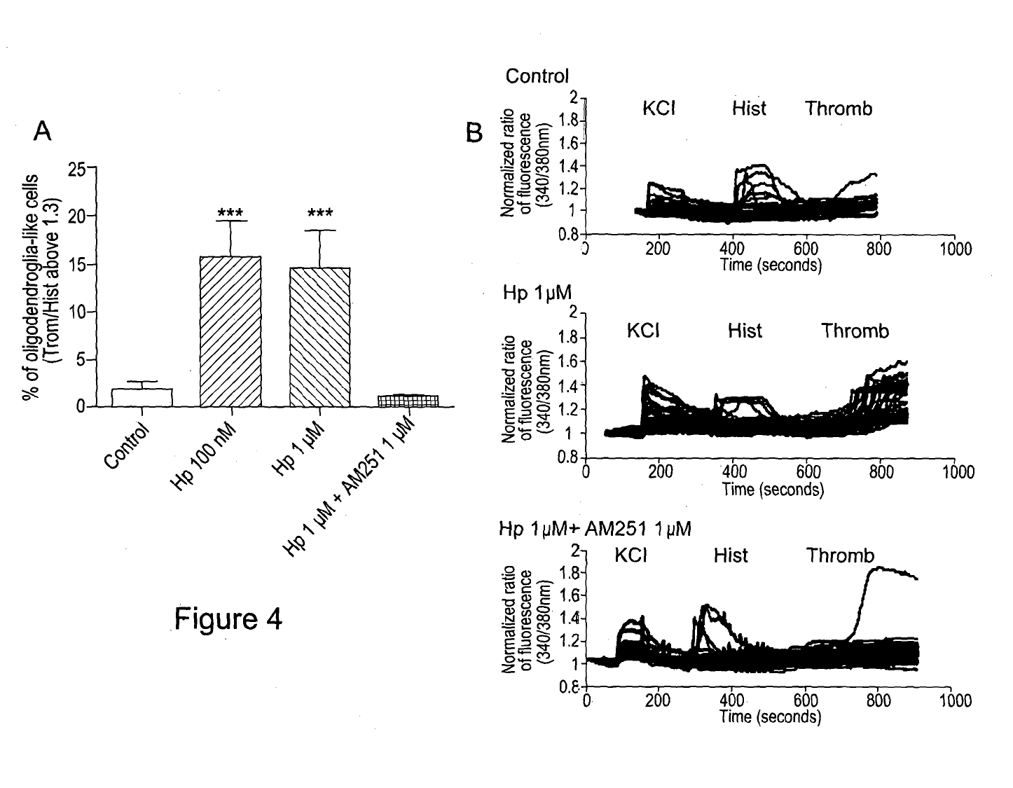

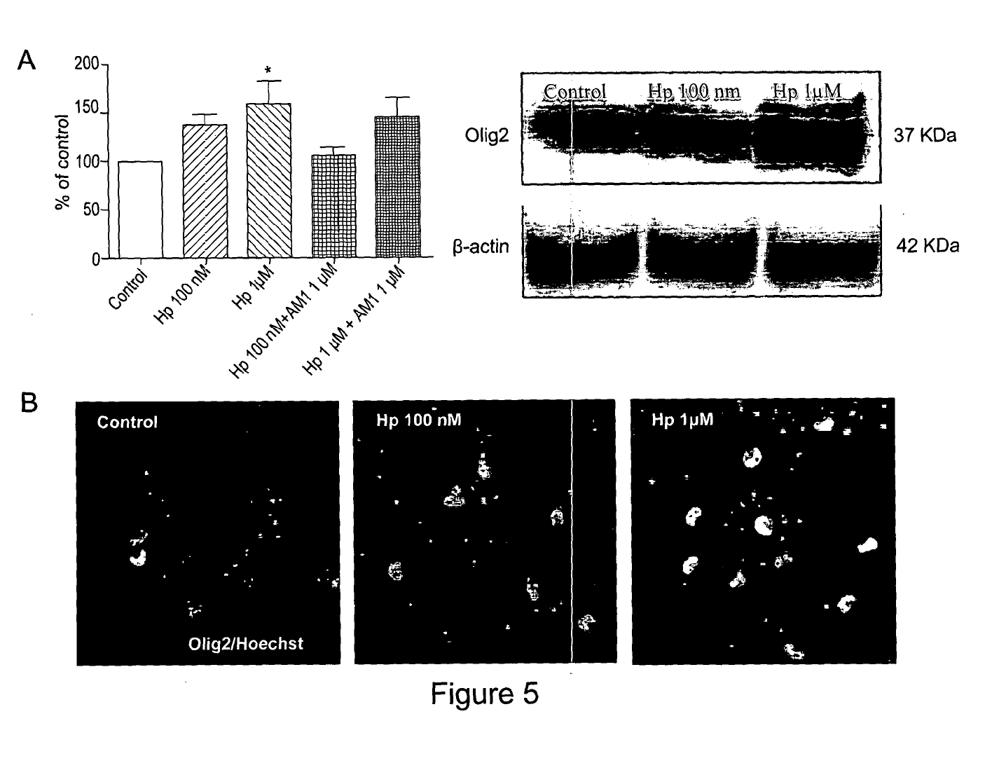

By using single-cell calcium imaging, based on cellular response to KCl, histamine and thrombin we were able to functionally evaluate oligodendroglial differentiation. Exposure of SVZ cultures to 100 nM or 1 μM Hp resulted in increased differentiation of cells displaying an oligodendrocyte-like profile of [Ca²⁺]_{I} responses, compared with the predominant profile of immature cells observed in non-treated control cultures. Moreover, by using an antagonist of CB₁ receptor we have observed that this effect is CB₁R dependent. Moreover, we performed immunocytochemistry and western blotting for Olig2 and we observed an increase in the immunoreactivity and protein levels when the cultures were exposed to Hp. The present invention thus demonstrates that SVZ cells express CB₁R (see FIG. 1). We further show (see FIG. 2) that Hp does not induce cell proliferation or cell death in subventricular zone (SVZ) cell cultures. In addition (see FIG. 3), we show that the number of primary neurospheres decreased in SVZ cell cultures grown in SFM containing 5 ng/ml EGF and 2.5 ng/ml FGF-2 and supplemented with 1 μM Hp. While Hp promoted SVZ cells capacity to self-renew when dissociated and replated without Hp, primary neurospheres that were exposed to Hp for 6 days generated higher numbers of secondary neurospheres in comparison with control SVZ cultures. Most significantly, we show (see FIGS. 4 and 5) that Hp induces oligodendrocyte differentiation in mouse SVZ cell cultures and increased Olig2 (37 KDa) production, while β-actin (42 KDa) protein levels remain unchanged in SVZ cultures exposed to Hp.

Taken together, these results show that Hp induces oligodendrogenesis in neonatal SVZ cell cultures of mice, demonstrating that Hp and related compounds disclosed herein are useful in strategies to treat or prevent demyelinating diseases. Moreover, synthetic compounds can cause severe side effects. For example, as shown in WO 2011/011847 which discloses the use of both the CB1R antagonist rimonabant and Hp in treating obesity, rimonabant can lead to unwanted site effects. As hemopressin peptides occur naturally in the brain, it is believed that SEQ ID No. 1 and its variants can have beneficial biological effects without inducing negative side effects of synthetic compounds.

Therefore, in a first aspect, the invention relates to an Hp peptide comprising or consisting of SEQ ID No. 1 or a variant thereof for use in the treatment of a neurodegenerative disorder.

As shown herein, an Hp peptide as defined in SEQ ID No. 1 or a variant thereof induces progenitor cells to differentiate into oligodendrocytes. The progenitor cells may, as discussed above, include any cells that can differentiate into oligodendrocytes, including OPCs, NSCs, NPCs and cells derived from the SVZ. The Hp peptide as defined in SEQ ID No. 1 or a variant thereof acts specifically on the endocannabinoid CB1 receptor to promote oligodendrogenesis.

Further, the Hp peptide as defined in SEQ ID No. 1 or a variant thereof does not increase cell death.

The term neurodegenerative disorder is understood to mean any disorder that affects oligodendrogenesis, differentiation of neural precursor cells into mature oligodendrocytes and conditions associated with demyelination or dysmyelination.

Diseases of the nervous system associated with demyelination or dysmyelination, include, but are not limited to MS, progressive multifocal leukoencephalopathy (PML), encephalomyelitis (EPL), central pontine myelolysis (CPM), Wallerian Degeneration, transverse myelitis, Devic's disease, Guillain-Barré syndrome, Charcot-Marie-Tooth disease, spinal cord injury, adrenoleukodystrophy, Alexander's disease and Pelizaeus Merzbacher disease (PMZ), Globoid cell Leucodystrophy (Krabbe's disease), optic neuritis, amylotrophic lateral sclerosis (ALS), Huntingtoris disease, Alzheimer's disease, Parkinson's disease, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, metachromatic leukodystrophy, trigeminal neuralgia, and Bell's palsy.

In one embodiment, the Hp peptide as defined in SEQ ID No. 1 or a variant thereof is for use in the treatment of MS. There is no known cure for demyelinating diseases such as MS and currently there is no effective treatment. In fact, total remyelination has so far not been reported. Moreover, the currently used medications can have adverse effects or be poorly tolerated. The present invention provides hemoglobin-derived or related peptides which exhibit pro-oligodendrogenic activity, which results in significant amelioration in demyelinating models and are thus useful in treating and/or preventing demyelination.

Medications for multiple sclerosis which are currently available include the following:
Corticosteroids: These affect immunologic actions, such as inflammation and immune responses. Corticosteroids are rarely used for a long time because they can have many side effects, such as increased susceptibility to infection, diabetes, weight gain, fatigue, decreased bone density (osteoporosis), and ulcers.
Immune-modulating or immunosuppressant drugs: These decrease the ability of the immune cells to cause inflammation. The most commonly reported side effects of these drugs are injection site disorders, flu-like symptoms, liver function loss, and blood cell abnormalities. Also, nausea, vomiting, heart damage, and immunosuppression may occur depending on the drug used. Recently, an antibody drug (Natalizumab (Tysabri), Biogen Idec) has been approved for treatment of MS, but the use of this drug carries a finite risk of inducing the often fatal iatrogenic disease Progressive Multifocal Leukoenkephalopathy in the patients (see, for example, Yousry et al., N Engl J. Med. 2006 March 2; 354(9): 924-933). Most of these drugs are delivered by frequent injections, varying from once-per-day to once-per-month.
Interferon beta-1a (Avonex from Biogen Idec, CinnoVex from CinnaGen and Rebif from EMD Serono Inc. and Pfizer Inc.)
Interferon beta-1b (Betaseron or Betaferon from Bayer Schering Pharma).
Glatiramer acetate (Copaxone from Teva Pharmaceutical Industries).
Mitoxantrone (Novantrone from EMD Serono).

The drugs currently available for the treatment of MS exercise their effect mainly on the inflammation, which is one of the causes of the demyelination. However, it is important to generate new drugs capable of inducing oligodendrogenesis and thus repairing myelin loss.

According to the present invention, any of these existing modes of therapy of a neurodegenerative disease may be supplemented or supplanted by the methods of treatment using the compounds and compositions of the present invention. The other drugs may form part of the same composition, or be provided as a separate composition for administration at the same time or a different time. Combination therapy using the present compositions, compounds and methods permits the negative impact of the existing therapies to be diminished or eliminated. Thus, the invention also relates a peptide according to SEQ Id No. 1 or a variant thereof for use in the treatment of MS wherein said peptide is administered in combination with a known therapy, for example any of the compounds listed above.

In another aspect of the invention, the Hp peptide of SEQ ID No. 1 or a variant thereof may be contacted in vitro/ex vivo with a neural progenitor cell that is capable of differentiating into an oligodendrocyte, such as a NPC, NSC, OPC, and the like, e.g. a cell derived from the SVZ, and the thus-treated cell may be implanted into a subject in need of such treatment. In a preferred embodiment, OPCs derived from the subject in need of such treatment are harvested from the subject, treated in vitro or ex vivo by exposure to the compound to initiate oligodendrocyte differentiation, and the thus treated cells are reimplanted into the subject at a site in need of such treatment. Such site may be in the brain, in the spinal cord, or a peripheral site, or combinations of such sites, where the subject is in need of such treatment (i.e. is suffering from a condition associated with loss of myelination).

Thus, the invention relates to a method of treatment comprising:
a) contacting a neural progenitor cell with peptide of SEQ ID No. 1 or a variant thereof in vitro,
b) allowing said cell to differentiate into an oligodendrocyte
c) administering said cell to a subject.

In another embodiment, the peptides of the invention may be directly injected into the desired location (i.e. the brain) to achieve localized increase in progenitor cell differentiation.

The compounds according to this invention include the Hp peptide and compounds that are related to Hp. The isolated Hp peptide has the amino acid sequence:

(SEQ. ID. 1)
Pro Val Asn Phe Lys Phe Leu Ser His,
Single letter code: PVNFKFLSH

The various aspects of the invention relate to SEQ ID No. 1 and variants thereof. The term variant/variants is understood to mean mimics, derivatives or fragments of SEQ ID No. 1 that have the same biological activity, that is selective binding to CB₁R and inducing oligodendrocyte progenitor cells to differentiate into oligodendrocytes.

Variant compounds for use in compositions and methods according to the present invention include but are not limited to:

SEQ. ID. 2.
Val Asp Pro Val Asn Phe Lys Phe Leu Ser His,
VDPVNFKFLSH
SEQ. ID. 3.
Arg Val Asp Pro Val Asn Phe Lys Phe Leu Ser His,
RVDPVNFKFLSH
SEQ ID. 4.
PVNFKWLSH,

When administered to rats, SEQ ID 4 has been shown in WO 2011/011847 to result in reduced periepididymal and visceral fat content when compared to the control group.

It is noted that there is a common peptide sequence in all of the three peptides, namely the SEQ. ID. 1. core sequence, PVNFKFLSH. Peptides exhibiting a sequence in which this core appears, albeit extended on the amino-terminal or carboxy-terminal end by a variety of amino acids are anticipated to exhibit the activity relevant to the present patent disclosure. Peptides which in total exhibit at least about 75% amino acid sequence homology with respect to any one of the SEQ ID. 2, 3 or 4 are likewise anticipated to exhibit activity of importance to the induction of oligodendrogenesis. It will be appreciated by those skilled in the art that while certain variants may exhibit subtly different properties, through routine experimentation based on the guidance provided herein, those skilled in the art are able to define compositions and modalities suited to treatment of a given pathologic condition.

Thus, in one aspect, a variant of SEQ ID No. 1 may be a peptide as defined in SEQ ID No. 2, 3 or 4. In another embodiment, a variant of SEQ ID No. 1 is a peptide which has at least about 75% to 95%, preferably at least 75%, 76%, 77%, 78%, 79%, 80%, 81%, 82%, 80%, 84%, 85%, 86%, 87%, 88%, 89%, 90%, 91%, 92%, 93%, 94% or 95% amino acid sequence homology with respect to SEQ ID. 1.

For example, the variant with the sequence homology as defined above may comprise amino acid substitutions that do not affect the biological function of the peptide. For example, in WO 2011/011847, binding studies of the most important portions of the hemopressin amino acid sequence to CB₁ receptors were conducted. It was determined that the two phenylalanines of the hemopressin sequence should have characteristics of hydrophobic and aromatic groups and the leucine portion of the hemopressin sequence should have characteristics of a hydrophobic group. Derivatives of hemopressin with hydrophobic and aromatic groups at the phenylalanine region and hydrophobic groups at the leucine region were shown to bind most successfully to CB′ receptors. It is therefore anticipated that the same considerations will apply to the present invention. Thus, in one embodiment, the variant of SEQ ID No. 1 may be a peptide wherein the two phenylalanines of the peptide of SEQ ID No. 1 are replaced by any two hydrophobic and aromatic groups.

In another embodiment, the variant of SEQ ID No. 1 may be a peptide wherein the leucine portion of the peptide of SEQ ID No. 1 is replaced by any hydrophobic group.

Other amino acid substitutions may also be introduced. A substitution mutation can be made to change an amino acid in a non-conservative manner or in a conservative manner. Such a conservative change generally leads to less change in the structure and function of the resulting protein and is preferred. A non-conservative change is more likely to alter the structure, activity or function of the resulting protein. The present invention should be considered to include peptide sequences containing conservative changes which do not significantly alter the activity or binding characteristics of the resulting protein.

Examples of substitutions are:

Arg for Lys such that a positive charge may be maintained;
Thr for Ser for such that a free —OH can be maintained; and
Gln for Asn such that a free NH2 can be maintained.

Without wishing to be bound by mechanistic considerations, it is believed that the compounds described herein and compositions comprising these compounds (which include a pharmaceutically acceptable vehicle) exert their pro-oligodendrogenesis effect by virtue of behaving as an agonist, inverse agonist and/or antagonist of a G protein-coupled receptor, in particular CB₁R. The compounds described herein are characterized in that administration of said pharmaceutical composition induces protenitor cells to differentiate into oligodendrocytes.

Of course, the prooligodendrogenic effect of these peptides on progenitor cells of relevance to the present invention may be characterized as that of an agonist, antagonist, inverse agonist or the like, provided that the compounds according to this invention exhibit the key desirable activity of inducing NPCs, OPCs, and the like, to differentiate into myelin producing cells which can ameliorate diseases associated with demyelination. Again, without wishing to be bound by mechanistic considerations, it is believed that the G protein-coupled receptor targeted in this invention is a cannabinoid receptor and in particular the CB₁ receptor.

In yet a further aspect of the invention, the peptides described in the different aspects of the invention can be formulated as pharmaceutical compositions with stabilizers to prevent proteolytic degradation, a pharmaceutically acceptable vehicle, carrier, diluent or excipient. Examples of pharmaceutical compositions include any solid (tablets, pills, capsules, granules, etc.) or liquid (solutions, suspensions or emulsions), and they may contain the pure compound or in combination with any carrier or other pharmacologically active compounds. These compositions may need to be sterile when administered parenterally. Pharmaceutical compositions containing compounds of the invention may be delivered by liposome or nanosphere encapsulation, in sustained release formulations or by other standard delivery means.

The pharmaceutically acceptable vehicle is preferably a sterile isosmotic solution with the same osmotic pressure of an isotonic solution of blood and that is compatible with the active ingredient.

The invention describes how said pharmaceutical composition may be used in the treatment of a neurodegenerative disease. The invention also reveals how said pharmaceutical composition may be used in the prevention and treatment of multiple sclerosis or other diseases associated with demyelination by inducing pluripotent or multipotent pre-oligodendrocytes to mature and differentiate into oligodendrocytes. Furthermore, this invention demonstrates that said pharmaceutical composition can effectively achieve said results through any suitable route of administration including but not limited to oral, transmucosal, intraperitoneal, intravenous, or intrathecal administration, and/or via an in vitro treatment of pre-oligodendrocytes which are then introduced into a subject in need of such treatment.

For oral administration, methods know to the skilled person to increase the intestinal absorption of the peptide by the use of formulations that protect the peptide from enzymatic degradation and/or enhance the uptake into the intestinal mucosa. For example, at least one absorption enhancer effective may be included in the pharmaceutical formulation comprising the peptide to promote bioavailability of said peptide. Also, an enteric coating may be included to mitigate against degradation in the digestive tract. Various types of commercial enteric coating polymers are candidates for coating materials to make the enteric coated particles or tablets, including aqueous dispersions or organic solutions of enteric polymers such as methacrylic acid co polymers (Eudragit L or S), cellulose acetate phthalate, cellulose acetate butyrate, hydroxypropyl methyl cellulose phthalate, polyvinyl acetate phthalate etc. Moreover, a coating may also be included to allow the drug to cross the blood brain barrier. Various coatings and techniques that facilitate this are available in the art.

Hemoglobin-derived peptides for use according to the present invention arise as pro-oligodendrogenic drugs and, being small molecules, are believed to cross the blood-brain-barrier (see WO 2011/011847) and therefore it is anticipated that these peptides may be administered centrally. The aspects of the invention use a therapeutically effective amount. Those skilled in the art, based on the present invention, are able to determine therapeutically effective amount and thus the appropriate dosages and treatment regimens without undue experimentation. Administration of a therapeutically effective amount of the peptides described herein to a patient or cells in vitro/ex vivo will result in oligodendrogenesis and myelination or remyelination as needed in a given physiologic condition.

For example, the peptide described herein may be administered in a dose of about 0.05 micrograms per kilogram of body weight to 1 milligram per kilogram of body weight, preferably at about 0.05 micrograms per kilogram of body weight to 50 micrograms per kilogram of body weight. However, the correct dosage of the compounds will vary according to the particular formulation, the mode of application, and the particular situs, host and condition being treated. Other factors like age, body weight, sex, diet, time of administration, rate of excretion, condition of the host, drug combinations, reaction sensitivities and severity of the disease shall be taken into account.

Further, the present invention contemplates treatment by gene therapy, where a nucleic acid encoding a peptide described herein is introduced into a target cell for treatment, to cause or increase expression of the corresponding peptide. Thus, in one embodiment, the nucleic acid is introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Suitable viral vectors for such gene therapy are known in the art.

Thus, the invention relates to expressing a nucleic acid sequence optionally operably linked to a control sequence encoding a peptide as defined in SEQ ID No. 1 in a host. The method comprises transforming or transfecting an implantable host cell with a nucleic acid, e.g., a vector that expresses a peptide of SEQ ID No. 1. The method may further comprise implanting or injecting the transformed host cell into a mammal, at the site of a disease, disorder or injury. For example, the transformed host cell can be implanted at the site of a chronic lesion of MS.

The invention also relates to the use of a peptide as defined in SEQ ID No. 1 or a variant thereof in the manufacture of a medicament for the treatment of a neurodegenerative disorder. The various embodiments of this use are as defined above.

The invention also relates to a method for treating a neurodegenerative disorder by administration of a peptide as defined in SEQ ID No. 1 or a variant thereof in the treatment of a neurodegenerative disorder. The various embodiments of this method are those described above with relation to a peptide as defined in SEQ ID No. 1 or a variant thereof for use in the treatment of a neurodegenerative disorder.

The invention also provides a medical kit for administration of a peptide comprising or consisting of SEQ ID No. 1 or a variant thereof comprising a supply of a peptide comprising or consisting of SEQ ID No. 1 or a variant thereof in a therapeutically effective dosage, a pharmaceutically acceptable carrier, and printed instructions for administering the peptide comprising or consisting of SEQ ID No. 1 or a variant thereof according to a dosing schedule.

### EXAMPLES

Although the present invention and its advantages have been described in detail herein above, it must be understood that various changes, substitutions and alterations may be made without straying from the core and scope of the invention as defined in the claims appended hereto.

Further, while the foregoing general description of this invention enables those skilled in the art to practice the present invention, the following examples are provided to extend the written description of this invention and to ensure that those skilled in the art are able to practice the full scope of this invention, as claimed herein, including its best mode. The specifics of the examples which follow are not, however, to be construed as limiting.

All experiments were performed in accordance with the European Community (86/609/EEC) guidelines for the care and use of laboratory animals and Portuguese legislation “Diário da República—Portaria 10005/92, 23 de Outubro”.

### Example 1

### SVZ Cultures

SVZ cell cultures were obtained from early postnatal (P1-3) C57B1/6 donor mice as described previously [1].

Brains were removed following decapitation and placed in HBSS solution (Gibco, Carlsbad, Calif., USA). Fragments of SVZ were dissected out of 450 μm thick coronal brain sections, digested in 0.025% trypsin and 0.265 mM EDTA (Gibco), and dissociated by gentle mixing. The cell suspension was diluted in serum-free culture medium (SFM) composed of Dulbecco's modified eagle medium (D-MEM/F12 Gluta-MAX™-I, Gibco) supplemented with 100 U/mL penicillin, 100 μg/mL streptomycin (Gibco), 1% B27 (Gibco), 10 ng/mL epidermal growth factor (EGF; Gibco), and 5 ng/mL basic fibroblast growth factor (FGF-2, Gibco). Single cells were then plated on uncoated Petri dishes at a density of 3000 cells/cm². The neurospheres were allowed to develop in a 95% air-5% CO₂ humidified atmosphere at 37° C.

Six- to 8-day-old neurospheres were adhered for 48 hours onto poly-D-lysine-coated glass coverslips in SFM devoid of growth factors. Then, to evaluate proliferation/cell death and neuronal differentiation, the neurospheres were allowed to develop for 48 hours or 7 days at 37° C. in the absence or in the presence of hemopressin (100 nM or 1 μM, Proteimax, Cotia, Brazil) and the CB1R antagonist AM 251 (1 μM, Tocris, Ellisville, Mo., USA).

### Example 2

### Immunocytochemistry

Cells were fixed for 30 minutes in 4% paraformaldehyde in phosphate-buffered saline (PBS), permeabilized and blocked for non-specific binding sites for 1 h with 0.25% Triton X-100 (Sigma) and 3% bovine serum albumin (BSA, Sigma) dissolved in PBS. Cells were then subsequently incubated overnight at 4° C. with the following antibodies: rabbit polyclonal anti-CB1R (1:200, Proteimax), mouse monoclonal anti-nestin (1:200, Chemicon, Temecula, Calif., USA), mouse monoclonal anti-GFAP (1:500, Cell Signaling Technology, Danvers, Mass., USA) and rabbit polyclonal anti-Olig2 (1:200, Millipore, Billerica, Mass., USA). Thereafter, the cover slips were rinsed in PBS and incubated for 1 h at RT with the appropriate secondary antibodies: anti-rabbit IgG labeled with Alexa Fluor 488 (1:200) or anti-mouse IgG labeled with Alexa Fluor 594 (1:200) (both from Molecular Probes). After rinsing with PBS, cell preparations were incubated 5 min at RT with Hoechst 33342 (2 μg/mL, Molecular Probes) in PBS, for nuclear staining. Finally, the preparations were mounted using Dakocytomation fluorescent medium (Dakocytomation, Carpinteria, Calif., USA). Fluorescence images were recorded using a digital camera coupled to an Axioskop microscope (Carl Zeiss).

### Example 3

### Apoptosis Assay

Cell apoptosis was evaluated by the terminal deoxynucleotidyl transferase dUTP nick-end labeling (TUNEL) assay which is a method that detects DNA fragmentation. Hence, this method is based on the specific activity of terminal transferase, which attaches labelled biotin-16-2′-deoxy-uridine-5′-triphosphate to the 3′-OH end of DNA generated during apoptotic-induced DNA fragmentation.

SVZ cells treated with Hp for 48 h, in serum free medium devoid of growth factor (differentiation conditions), were rinsed 3×10 min with 0.15 M PBS and permeabilized in 0.25% Triton X-100 for 30 min at RT. Cells were then incubated with terminal deoxynucleotidyl transferase buffer (0.25 U/μl terminal transferase), 6 μM biotinylated dUTP, pH 7.5)) (all from Roche, Basel, Switzerland) for 1 h at 37° C. in a humidified chamber, and then rinsed in TB buffer (300 mM NaCl and 30 mM sodium citrate) for 15 min and in PBS for 5 min. Incubation with Fluorescein was performed for 1 h and nuclei counterstaining and mounting were performed as described previously.

### Example 4

### SVZ Neurosphere Forming and Self-Renewal Assay

Neurosphere forming and self-renewal assays were performed on SVZ cells seeded at clonal density, 2500 cells per well in 24-well cell culture plates in SFM containing 5 ng/ml EGF and 2.5 ng/ml FGF-2 and supplemented or not (control) with 1 μM Hp or 50 ng.ml⁻¹ hepatocyte growth factor (carrier free recombinant human HGF, R&D Systems, Lille, France). After 6 days, the numbers of primary neurospheres were determined. For self-renewal assay, neurospheres were collected, dissociated as single cells and seeded in SFM containing 5 ng/ml EGF and 2.5 ng/ml FGF-2 in 24 well plates. After 6 days, the number of secondary neurospheres was counted.

### Example 5

### Cell Proliferation Studies

To investigate the effect of Hp on cell proliferation, SVZ cells were exposed to 10 μM 5-bromo-2′-deoxyuridine (BrdU) (Sigma-Aldrich) for the last 4 hours of each Hp—treatment (48 hours). Then, SVZ cells were fixed in 4% PFA for 30 minutes and rinsed for 30 minutes in 0.15 M PBS at RT. SVZ cells were rinsed in PBS, thereafter BrdU was unmasked by permeabilizing cells in PBS 1% Triton X-100 at RT for 30 min and DNA was denaturated int M HCl for 40 min at 37° C. Following incubation in PBS with 0.5% TritonX-100 and 3% BSA to block nonspecific binding sites, cells were incubated overnight the primary mouse Alexa Fluor 594-conjugated monoclonal anti-BrdU antibody (1:100, Invitrogen, Carlsbad, Calif., USA). After an additional rinse in PBS, SVZ Nuclei counterstaining and mounting were performed as described previously.

### Example 6

### Single Cell Calcium Imaging (SCCI)

To investigate the influence of Hp on oligodendrocytic differentiation, SVZ neurospheres were allowed to develop for 7 days Hp (100 nm, 1 μM), in serum free medium devoid of growth factor at 37° C.

To determine the functional differentiation pattern of SVZ cells, we analyzed the variations of intracellular calcium-free levels ([Ca²⁺]ᵢ) in single cells following stimulation with 50 mM KCl, 100 μM histamine (Sigma-Aldrich, St. Louis, Mo. USA) and 0.1 U/ml thrombin (Sigma-Aldrich) stimulation. KCl depolarization causes an increase in [Ca²⁺]ᵢ in neurons, whereas stimulation with histamine leads to an increase in [Ca²⁺]ᵢ in stem/progenitor cells, while thrombin leads to an increase in [Ca²⁺]ᵢ in oligodendrocytes [2, 14]. SVZ cultures were loaded for 40 minutes with 5 μM Fura—2/AM (Molecular Probes, Carlsbad, Calif., USA), 0.1% fatty acid-free BSA, and 0.02% pluronic acid F-127 (Molecular Probes) in Krebs solution (132 mM NaCl, 4 mM KCl, 1.4 mM MgCl₂, 1 mM CaCl₂, 6 mM glucose, 10 mM HEPES, pH 7.4), in an incubator with 5% CO2 and 95% atmospheric air at 37° C. After a 10-minute postloading period at room temperature (RT) in the same medium without Fura-2/AM and pluronic acid, to obtain a complete hydrolysis of the probe, the glass coverslip was mounted on an RC-20 chamber in a PH3 platform (Warner Instruments, Hamden, Conn., USA) on the stage of an inverted fluorescence microscope (Axiovert 200, Carl Zeiss). Cells were continuously perfused with Krebs solution and stimulated at defined periods of time by applying high-potassium Krebs solution (containing 50 mM KCl, isosmotic substitution with NaCl), 100 μM histamine and 0.1 U/ml thrombin. Solutions were added to the cells by a fast-pressurized (95% air, 5% CO2 atmosphere) system (AutoMate Scientific, Inc., Berke-ley, CA, USA). The variations of [Ca²⁺]ᵢ were evaluated by quantifying the ratio of the fluorescence emitted at 510 nm following alternate excitation (750 msec) at 340 and 380 nm, using a Lambda DG4 apparatus (Sutter Instrument, Novato, Calif., USA) and a 510 nm band-pass filter (Carl Zeiss) before fluorescence acquisition with a x40 objective and a CoolSNAP digital camera (Roper Scientific, Trenton, N.J., USA). Acquired values were processed using the MetaFluor software (Universal Imaging Corp., West Chester, Pa., USA). KCl, histamine, and thrombin peaks given by the normalized ratios of fluorescence at 340/380 nm, at the proper time periods, were used to calculate the ratios of the responses to histamine/KCl (Hist/KCl-neuronal-like profile) and to thrombin/histamine (Throm/Hist-oligodendrocytic-like profile).

### Example 7

### Western Blotting Analysis

Western blotting analysis of Olig2 was performed from 6- to 8-days-old neurospheres that were plated onto six-well plates previously coated with poly-D-lysine, and that were allowed to adhere for 48 h in the presence of SFM, and treated in the absence (control) or in the presence of Hp and/or AM251 for 7 days. For the evaluation of Olig2 protein levels in control versus Hp-treated condition, the medium was renewed after 48 h. Seven days after the first treatment, the cells were washed with 0.15M phosphate-buffered saline (PBS) and harvested by scraping in the lysis buffer [0.15 M NaCl, 0.05 M Tris-base, 5 mM EGTA, 1% Triton X-100, 0.5% DOC, 0.1% SDS, 10 mM dithiothreitol (DTT), containing a protease inhibitor cocktail tablet (Roche Diagnostics GmbH, Germany), pH7.4 at 4° C.].

The supernatant was collected after centrifugation at 14 000 rpm for 10 min, at 4° C. Protein concentration was measured by the BCA method and samples were treated with SDS-PAGE sample buffer [6× concentrated: 350 mM Tris, 10% (w/v) SDS, 30% (v/v) glycerol, 0.6 M DTT, 0.06% (w/v) bromophenol blue], boiled 5 min at 95° C., and stored at −20° C. until use for Western blotting analysis. Then, proteins (50 μg of total protein) were separated by SDS-PAGE on 10% acrylamide/bisacrylamide gels and transferred onto PVDF (polyvinylidine difluoride) membranes with 0.45 μm pore size in the following conditions: 300 mA, 90 min at 4° C. in a solution containing 10 mM CAPS and 10% methanol, pH 11. Membranes were blocked in Tris buffer saline (TBS) containing 5% low-fat milk and 0.1% Tween 20 (Sigma) for 1 h at RT and then incubated overnight at 4° C. with the primary rabbit polyclonal anti-Olig2 antibody (1:200) (Millipore) diluted in 1% TBS-Tween and 0.5% low-fat milk. After rinsing three times with TBS-T 0.5% low-fat milk, membranes were incubated for 1 h at RT, with an alkaline phosphatase-linked secondary antibody anti-rabbit IgG 1:20 000 in 1% TBS-T and 0.5% low-fat milk (GE Healthcare, Buckingham-shire, UK). For endogenous control of immunolabeling, PVDF membranes were reprobed with the mouse monoclonal anti-β actin primary antibody (1:2000, Sigma-Aldrich) and with the alkaline phosphatase-linked anti-mouse secondary antibody (1:20 000, GE Healthcare).

Protein immunoreactive bands were visualized in a Versa-Doc Imaging System (model 3000, BioRad Laboratories, Calif.), following incubation of the membrane with ECF reagent (GE Healthcare, Buckinghamshire, UK) for 5 min. Densitometric analyses were performed by using the ImageQuant software.

### Example 8

### Statistical Analysis

Fluorescence images were recorded using an LSM 510 Meta confocal microscope or an Axioskop 2 Plus fluorescence microscope (both from Carl Zeiss). In all experiments, measurements were performed in the border of SVZ neurospheres where migrating cells form a cell monolayer. For the SVZ neurosphere forming and self-renewal assays the experiments were replicated in five independent culture preparations and each experimental condition was assayed in four different coverslips. For the remaining experiments, each condition was assayed in three different coverslips, and except where otherwise specified, the experiments were replicated in three independent culture preparations. Percentages of TUNEL or BrdU immunoreactive cells in SVZ cell cultures were calculated from cell counts in five independent microscopic fields in each coverslip with a x40 objective (approximately 200 cells per field). Software used was Axiovision, release 4.6 (Carl Zeiss). For SCCl experiments, the percentage of oligodendrocytic-like responding cells (with a Thromb/Hist ratio above 1.3) was calculated on the basis of one microscopic field per coverslip, containing approximately 100 cells (magnification, ×40).

Data are expressed as means±standard error of the mean (SEM). Statistical significance was determined by using the unpaired two-tailed Student's t test or one-way analysis of variance followed by Dunnett's-multiple comparasion test, with p<0.05 considered to represent statistical significance.

### Example 9

### SVZ Cells Express CB1R

To disclose whether CB1R is expressed on differenting cells, SVZ neurospheres were seeded onto poly-D-lysine and allowed to differentiate in SFM devoid of growth factors for 7 days. During this period of time, cells migrate out of the neurospheres and form a pseudo-monolayer so-called “carpet,” constituted of neurons, oligodendrocytes, and astrocytes in different stages of maturation. CB1R was detected by immunocytochemistry in nestin-positive (FIG. 1A) SVZ cells and in GFAP-positive astrocytes (FIG. 1B), showing that CB1R are localized in neural progenitor cells.

### Example 10

Hp does not Promote SVZ Cell Proliferation and Exerts No Effect on Cell Death

To investigate the effect of Hp on proliferation, SVZ neurospheres derived from newborn mice were treated for 48 hours in the absence (control condition) or presence of Hp. The thymidine analogue BrdU that incorporates in DNA in S-phase of the cell cycle was added for the last 4 hours of both culture sessions. Nuclei were then immunostained for BrdU, as shown in FIG. 2A. Hp does not induce a significant increase in the percentage of BrdU-positive cells when compared with control cells (control 4.77±0.25%, Hp 100 nM 4.01±0.81%; Ho 1 μM 4.70±0.53%; FIG. 2B).

The effect of Hp on cell death was evaluated after 48 hours of treatment with Hp. Apoptotic nuclei were stained by the TUNEL method (FIG. 2C) and no significant differences in the numbers of TUNEL-positive nuclei were found, indicating that Hp is not toxic to the cells (control 11.38%±1.14%; Hp 100 nM 10.26±1.69, Hp 1 μM 13.25±1.56%, FIG. 2D).

### Example 11

### Hp Promotes Self-Renewal in SVZ Cell Cultures

Neural stem-like cells are characterized in vitro by both their capacity to give rise to neurospheres and to self-renew when cultured in the presence of mitogens [26]. Exposure of SVZ cultures to 1 μm Hp during 6 days decreased by 15% the number of primary neurospheres (control: 100±2.90%, Hp 1 μM: 85.13±3.86%, HGF 50 ng.ml⁻¹: 121.0±13.14%, FIG. 3A), while HGF used here as a positive control [20], increase the number of primary neurospheres. However, Hp or HGF promoted SVZ cells capacity to self-renew as, when dissociated and replated without Hp or HGF, primary neurospheres that were exposed to Hp or HGF for 6 days generated higher numbers of secondary neurospheres in comparison with control SVZ cultures (control: 100±3.09%, Hp 1 μM: 125.5±13.38%, HGF 50 ng.ml⁻¹: 120.0±11.54%, FIG. 3B).

### Example 12

### Hp Induces Oligodendroglial Differentiation

To investigate whether Hp might influence the capacity of SVZ cells to differentiate into functional oligodendrocytes, 6-8-day-old SVZ neurospheres were allowed to develop on poly-D-lysine-coated coverslips for 7 days in the presence of Hp (100 nM or 1 μM). At the border of neurospheres, migrating cells emerged, forming a cell monolayer, where all the measurements of [Ca^{2+]}_{I} and cell countings of immunostainings were performed. At the end of Hp treatments, the SVZ cells were loaded with the Fura-2/AM calcium probe, perfused continuously for 15 minutes with Krebs solution, and briefly (2 min) stimulated with 50 mM KCl, 100 μM histamine, or with 0.1 U/ml thrombin.

Upon SCCI assay consisting of KCl, histamine, and thrombin pulses, normalized peaks of fluorescence of all the individualized cells were measured and the Throm/Hist ratio was calculated. Indeed, control cultures presented around 2% of cells responding with a Throm/Hist ratio above 1.3 (1.88±0.79%, 21 coverslips) consistent with the normal oligodendrocyte differentiation in SVZ cultures. Upon Hp treatment, cultures contained around 15% of these cells (Hp 100 nM: 15.84±3.85%, 9 coverslips; Hp 1 μM: 14.51±4.09%, 12 coverslips) (FIG. 4). Moreover, the CB1R antagonist Am 251 (1 μM) blocked Hp effect on oligodendrocytic differentiation (1.07±0.24%, 8 coverslips) (FIG. 4).

Moreover, we performed western blotting and immunocytochemistry for Olig2 and we observed an increase in Olig2 protein levels under Hp treatment (Hp 100 nM: 136.9±10.94, Hp 1 μM: 158.7±23.62, n=8) when compared to control (100%) (FIG. 5A). Moreover, the number of Olig2-positive cells observed by immunocytochemistry increased under Hp treatment (FIG. 5B).

### REFERENCES

1. Agasse F, Bernardino L, Kristiansen H, Christiansen S H, Ferreira R, Silva B, Grade S, Woldbye D P, Malva J O (2008) Neuropeptide Y promotes neurogenesis in murine subventricular zone. Stem Cells 26:1636-1645
2. Agasse F, Bernardino L, Silva B, Ferreira R, Grade S, Malva J O (2008) Response to histamine allows the functional identification of neuronal progenitors, neurons, astrocytes, and immature cells in subventricular zone cell cultures. Rejuvenation Res 11:187-200
3. Akiyama Y, Honmou O, Kato T, Uede T, Hashi K, Kocsis J D (2001) Transplantation of clonal neural precursor cells derived from adult human brain establishes functional peripheral myelin in the rat spinal cord. Exp Neurol 167:27-39
4. Arevalo-Martin A, Garcia-Ovejero D, Rubio-Araiz A, Gomez O, Molina-Holgado F, Molina-Holgado E (2007) Cannabinoids modulate Olig2 and polysialylated neural cell adhesion molecule expression in the subventricular zone of post-natal rats through cannabinoid receptor 1 and cannabinoid receptor 2. Eur J Neurosci 26:1548-1559
5. Betarbet R, Zigova T, Bakay R A, Luskin M B (1996) Dopaminergic and GABAergic interneurons of the olfactory bulb are derived from the neonatal subventricular zone. Int J Dev Neurosci 14:921-930
6. Biagioli M, Pinto M, Cesselli D, Zaninello M, Lazarevic D, Roncaglia P, Simone R, Vlachouli C, Plessy C, Bertin N, Beltrami A, Kobayashi K, Gallo V, Santoro C, Ferrer I, Rivella S, Beltrami C A, Carninci P, Raviola E, Gustincich S (2009) Unexpected expression of alpha- and beta-globin in mesencephalic dopaminergic neurons and glial cells. Proc Natl Acad Sci USA 106:15454-15459
7. Cayre M, Bancila M, Virard I, Borges A, Durbec P (2006) Migrating and myelinating potential of subventricular zone neural progenitor cells in white matter tracts of the adult rodent brain. Mol Cell Neurosci 31:748-758
8. Dodd G T, Mancini G, Lutz B, Luckman S M (2010) The peptide hemopressin acts through CB1 cannabinoid receptors to reduce food intake in rats and mice. J Neurosci 30:7369-7376
9. Franklin R J, Ffrench-Constant C (2008) Remyelination in the CNS: from biology to therapy. Nat Rev Neurosci 9:839-855
10. Galve-Roperh I, Aguado T, Palazuelos J, Guzman M (2008) Mechanisms of control of neuron survival by the endocannabinoid system. Curr Pharm Des 14:2279-2288
11. Gomes I, Grushko J S, Golebiewska U, Hoogendoorn S, Gupta A, Heimann A S, Ferro E S, Scarlata S, Fricker L D, Devi L A (2009) Novel endogenous peptide agonists of cannabinoid receptors. FASEB J 23:3020-3029
12. Gomez O, revalo-Martin A, Garcia-Ovejero D, Ortega-Gutierrez S, Cisneros J A, Almazan G, Sanchez-Rodriguez M A, Molina-Holgado F, Molina-Holgado E (2010) The constitutive production of the endocannabinoid 2-arachidonoylglycerol participates in oligodendrocyte differentiation. Glia 58:1913-1927
13. Gonzalez-Perez O, varez-Buylla A (2011) Oligodendrogenesis in the subventricular zone and the role of epidermal growth factor. Brain Res Rev
14. Grade S, Agasse F, Bernardino L, Silva C G, Cortes L, Malva J O (2010) Functional identification of neural stem cell-derived oligodendrocytes by means of calcium transients elicited by thrombin. Rejuvenation Res 13:27-37
15. Heimann A S, Gomes I, Dale C S, Pagano R L, Gupta A, de Souza L L, Luchessi A D, Castro L M, Giorgi R, Rioli V, Ferro E S, Devi L A (2007) Hemopressin is an inverse agonist of CB1 cannabinoid receptors. Proc Natl Acad Sci USA 104:20588-20593
16. Lois C, Alvarez-Buylla A (1994) Long-distance neuronal migration in the adult mammalian brain. Science 264:1145-1148
17. Lois C, Garcia-Verdugo J M, Alvarez-Buylla A (1996) Chain migration of neuronal precursors. Science 271:978-981
18. Menn B, Garcia-Verdugo J M, Yaschine C, Gonzalez-Perez O, Rowitch D, varez-Buylla A (2006) Origin of oligodendrocytes in the subventricular zone of the adult brain. J Neurosci 26:7907-7918
19. Nait-Oumesmar B, Picard-Riera N, Kerninon C, Baron-Van E A (2008) The role of SVZ-derived neural precursors in demyelinating diseases: from animal models to multiple sclerosis. J Neurol Sci 265:26-31
20. Nicoleau C, Benzakour O, Agasse F, Thiriet N, Petit J, Prestoz L, Roger M, Jaber M, Coronas V (2009) Endogenous hepatocyte growth factor is a niche signal for subventricular zone neural stem cell amplification and self-renewal. Stem Cells 27:408-419
21. Picard-Riera N, Nait-Oumesmar B, Baron-Van E A (2004) Endogenous adult neural stem cells: limits and potential to repair the injured central nervous system. J Neurosci Res 76:223-231
22. Pluchino S, Quattrini A, Brambilla E, Gritti A, Salani G, Dina G, Galli R, Del C U, Amadio S, Bergami A, Furlan R, Comi G, Vescovi A L, Martino G (2003) Injection of adult neurospheres induces recovery in a chronic model of multiple sclerosis. Nature 422:688-694
23. Richter F, Meurers B H, Zhu C, Medvedeva V P, Chesselet M F (2009) Neurons express hemoglobin alpha- and beta-chains in rat and human brains. J Comp Neurol 515:538-547
24. Smith P M, Blakemore W F (2000) Porcine neural progenitors require commitment to the oligodendrocyte lineage prior to transplantation in order to achieve significant remyelination of demyelinated lesions in the adult CNS. Eur J Neurosci 12:2414-2424
25. Solbrig M V, Fan Y, Hermanowicz N, Morgese M G, Giuffrida A (2010) A synthetic cannabinoid agonist promotes oligodendrogliogenesis during viral encephalitis in rats. Exp Neurol 226:231-241
26. Weiss S, Dunne C, Hewson J, Wohl C, Wheatley M, Peterson A C, Reynolds B A (1996) Multipotent CNS stem cells are present in the adult mammalian spinal cord and ventricular neuroaxis. J Neurosci 16:7599-7609
27. Imitola, Chitnis, Samia and Khoury (2006) Insights Into the Molecular Pathogenesis of Progression in Multiple Sclerosis 2006 ARCH NEUROL/VOL 63 63:25-33.
28. Gomes et al., “Hemoglobin-derived Peptides as Novel Type of Bioactive Signaling Molecules”, AAPS J., Vol. 12, No. 4, December 2010, pp. 658-669

1. A peptide according to SEQ ID NO:1 or a variant thereof.

2. The method of claim 16, wherein the neurodegenerative disorder affects oligodendrogenesis, differentiation of neural precursor cells into mature oligodendrocytes, or myelination.

3. The method of claim 2, wherein the neurodegenerative disorder is a condition associated with demyelination or dysmyelination.

4. The method of claim 16, wherein the neurodegenerative disorder is selected from the group consisting of multiple sclerosis (MS), progressive multifocal leukoencephalopathy (PML), encephalomyelitis (EPL), central pontine myelolysis (CPM), Wallerian Degeneration, transverse myelitis, Devic's disease, Guillain-Barre syndrome, Charcot-Marie-Tooth disease, spinal cord injury, adrenoleukodystrophy, Alexander's disease and Pelizaeus Merzbacher disease (PMZ), Globoid cell Leucodystrophy (Krabbe's disease), optic neuritis, amylotrophic lateral sclerosis (ALS), Huntingtoris disease, Alzheimer's disease, Parkinson's disease, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, metachromatic leukodystrophy, trigeminal neuralgia, and Bell's palsy.

5. (canceled)

6. The peptide of claim 1, wherein the peptide is a variant of SEQ ID NO:1 that has at least about 75%, 80%, 85%, 90% or 95% amino acid sequence homology with respect to SEQ ID NO:1.

7. The peptide of claim 6, wherein two phenylalanines of the peptide of SEQ ID NO:1 are replaced by any two hydrophobic and aromatic groups.

8. The peptide of claim 6, wherein the leucine of the peptide is replaced by any hydrophobic group.

9. The peptide of claim 1, wherein the variant is SEQ ID NO:1, SEQ ID NO:2, or SEQ ID NO:3.

10. The peptide of claim 1, wherein the peptide is formulated to be administered intraperitoneally, intrathecal, intravenously or orally.

11. The peptide of claim 1, wherein the peptide is formulated to be administered in a dose of about 0.05 micrograms per kilogram of body weight to 1 milligram per kilogram of body weight.

12. The method of peptide of claim 11, wherein the peptide is formulated to be administered in a dose of about 0.05 micrograms per kilogram of body weight to 50 micrograms per kilogram of body weight.

13. A method of treating a neurodegenerative disorder, the method comprising contacting the peptide of claim 1 ex vivo with a neural precursor cell (NPC), and implanting the NPC into a host at the site of a disease, disorder or injury to induce myelination or remyelination.

14. The method of claim 13, wherein the peptide that contacts the NPC is at a concentration of between about 1 nM and about 10 micro-molar.

15. (canceled)

16. A method of treating or preventing a neurodegenerative disorder, the method comprising administering to a subject in need of such treatment a pharmaceutically effective amount of a peptide of claim 1.

17. The method of claim 16, the method comprising exposing subventricular zone (SVZ) cells to 100 nM or 1 μM of the peptide to induce increased differentiation of cells displaying an oligodendrocytic-like profile of [Ca2+]_{I} responses, compared with the predominant profile of immature cells observed in control, non-treated cells.

18.-19. (canceled)

20. A method for inducing oligodendrocyte progenitor cells to differentiate into oligodendrocytes, the method comprising contacting the progenitor cells with an effective amount of a peptide of claim 1.

21. The method of claim 20, wherein contacting the progenitor cells occurs in vivo or ex-vivo.

22.-23. (canceled)

24. A pharmaceutical composition comprising the peptide of claim 1.

25. A medical kit comprising a supply of a peptide of claim 1 in a therapeutically effective dosage, a pharmaceutically acceptable carrier, and printed instructions for administering the peptide according to a dosing schedule.
